(19) United States
(12) Patent Application Publication
(10) Pub. No.: US 2008/0039705 A1
(43) Pub. Date: Feb. 14, 2008

(54) Map based intuitive device control and sensing to navigate a medical device

(76) Inventor: Raju R. Viswanathan, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 11/789,799

(22) Filed: Apr. 25, 2007

Related U.S. Application Data
(60) Provisional application No. 60/797,253, filed on May 3, 2006.

(51) Int. Cl. A61B 5/00

(52) U.S. Cl. 600/407

(57) ABSTRACT

A method is provided for adjusting a medical device given an image of the device position relative to an anatomical surface shown on a two-dimensional display, that includes using a user-input device to indicate a direction of adjustment of the device relative to the display.

### CROSS-REFERENCE TO RELATED APPLICATIONS

This application claims benefit of U.S. Provisional Patent Application Ser. No. 60/797,253, filed May 3, 2006, the entire disclosure of which is incorporated by reference.

### FIELD

This invention relates to remote surgical navigation, and more specifically to methods for controlling navigation of medical devices within a subject's body.

### BACKGROUND

One example of a control system for controlling a medical device within a subject's body is a magnetic navigation system where the medical device contains magnetic material that interacts with an externally applied magnetic field that is applied to suitably orient the device, for example the commercially available Stereotaxis Niobe magnetic navigation system. An alternative means of remote control of a medical device is an electromechanical system that uses servo-motors and cables to actuate the distal portion of the medical device within a subject, either directly or indirectly though actuating a sheath through which the medical device itself passes.

A specific example where fine control of medical device placement is required is in Electrophysiology procedures involving intracardiac ECG mapping and RF ablation for treatment of cardiac arrhythmias. These procedures deliver therapy by forming a lesion line of ablation spots where RF energy is delivered to destroy diseased tissue and restore normal electrical activity of the heart. The lesion line needs to be carefully and precisely formed and fine control of catheter movement is an important part of this process.

### SUMMARY

The present invention relates to directing a medical device within a subject and steering the device in a user-defined manner with respect to an anatomical map with a remote navigation system. In one embodiment, a method for adjustably moving a medical device relative to an anatomical surface being displayed on a display device is provided. The method comprises using a user-input device to select a point at or near the tip of a medical device depicted in an image on a two-dimensional display, and moving the user-input device a desired length in a desired adjustment direction relative to the point selected by the user on the displayed image. The method proceeds in displaying on the displayed image a line having a first endpoint corresponding to the user-selected point, and a direction and length corresponding to the length of movement of the user-input device in the desired adjustment direction. The method provides for determining a surface normal at a location corresponding to the point selected by the user, and determining a two-dimensional vector m corresponding to the line displayed on the display image. The method may employ an algorithm, for the purpose of determining a three-dimensional vector p in the tangent plane perpendicular to the surface normal, whose two-dimensional projection on the two-dimensional displayed image yields the two-dimensional vector m. The method may then determine a rotation of the device tip in the plane formed by a vector t representing the device tip's initial orientation and the three-dimensional vector p, which rotation corresponds to the desired adjustment movement. Where a magnetic navigation system is employed, the method includes rotating an external magnetic field about a vector normal to the plane of rotation formed by vector t and vector p, for causing the tip of the medical device to be moved in the desired adjustment direction by the magnetic navigation system.

In another aspect of the present invention, various embodiments of a method for controlling adjustments with a magnetic navigation system include applying an amount of magnetic field rotation in a fixed step size. Some embodiments for controlling adjustments with a magnetic navigation system include applying an amount of magnetic field rotation that depends on the length of the vector m representing the length of the movement of the user-input device.

In another aspect of the present invention, some embodiments of a method include updating the image being displayed on the display to show the real-time position of the medical device after an adjustment direction and length have been selected using the user-input device.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a view of a medical device near an anatomical surface within a subject's body, being controlled according to one embodiment of a method for adjusting the position of a medical device.

### DETAILED DESCRIPTION OF THE EMBODIMENTS

The following description of the various embodiments is merely exemplary in nature and is in no way intended to limit the invention, its application, or uses.

The present invention relates to directing a medical device within a subject and steering the device in a user-defined manner with respect to an anatomical map with a remote navigation system. The anatomical map is an object in three dimensions and could be a volume or surface data derived from pre-operative or intra-operative imaging. Alternatively, it could be a map reconstructed from a set of locations that have been visited by the tip of a medical device, or a surface map generated from recordings of electrical activity such as a set of intracardiac recordings. Remote navigation systems with device control as described in this invention may, for example, offer a new method for creating a lesion line of ablation spots to restore normal electrical activity of the heart.

In one embodiment, the anatomical map is generated by a localization system using a localized device navigated in a cardiac chamber. The map is a surface reconstruction generated from interpolation through a set of interior surface points on the inner cardiac wall that the tip of the medical device (typically a catheter) is guided to. The device tip is localized in real time and its known location and orientation are used to render a graphical catheter tip on the localization system display. An example is the CARTO™ system commercially available from Biosense Webster, Inc. In this embodiment, the localization system is integrated with a remote navigational system such that the medical device tip information and map-based information is sent to the remote navigation system.

In one embodiment utilizing a localization system, the catheter tip is visible on the localization display together with the anatomical map. When it is desired to adjust or modify the catheter or device tip location, the user selects a mode on the localization display. This mode selection permits the input of navigational commands to the remote navigation system as follows. The user uses a mouse or other user-input device to click or select a point at location x on the anatomical map that is nearest to the device tip. The user drags the mouse (or other user-input device) in a desired adjustment direction with reference to the map surface. A line is displayed on the localization display corresponding to the movement of the user dragging the mouse, to permit visualization of the adjustment direction. Such a “rubber-band” line moves with the mouse, so that easy adjustment becomes possible. When the user releases the mouse button, or selects a desired end point, a control variable is applied by the remote navigation system that actuates the device to make an adjustment in the direction indicated by the user. For this to occur seamlessly, certain variables are transferred from the localization system to the remote navigation system. These variables include the outward surface normal n at the initial location x that is the first endpoint of the line, the two-dimensional direction and length of the mouse drag represented as a two-dimensional vector m₂, the orientation (represented by a rotation matrix R) or the display view on the localization system with reference to a known, predetermined coordinate system relative to the subject, and the real-time device tip orientation t.

One control algorithm that may be utilized in combination with the above variables works in response to the user-input of an adjustment as follows:

As soon as the mouse button is released, thereby defining the desired adjustment direction, a three-dimensional vector p is computed as follows:

The view orientation rotation matrix R describes how the object in the view or displayed image is oriented. Thus, a vector u′ in a known fixed frame moves to a vector u as a result of the rotation:

u′=Ru (1)

The normal to the display screen or image is written as v′=(0, 0, 1), and corresponds to a three-dimensional view vector v=R^{T}v′ with respect to the object (vectors here are written as column vectors, and the superscript “^{T}” denotes a matrix transpose).

The vector m₂=(a, b) in the screen or displayed image coordinates may be represented as a three-dimensional vector m₃=(a, b, 0), corresponding to a vector m=R^{T} m₃ with respect to the object. The movement direction desired by the user can be thought of as an adjustment in a direction locally tangential to the surface, as this is the most natural adjustment of a medical device near a surface. Accordingly, we look for a three-dimensional vector p in the tangent plane perpendicular to the surface normal n, whose projection in the viewing plane yields the vector m. Mathematically, this yields the equations:

p·n=0 (2)

and

(I−vv^{T})p=m (3)

for the vector p, where I is the 3×3 identity matrix. Let c and d be any two (distinct) vectors orthogonal to v. From equation (3) we get:

c·p=m·c (4)

and

d·p=m·d (5)

Equations (2), (4) and (5) can be solved for the three-dimensional vector p.

If t is the initial device tip orientation, the movement indicated by p corresponds to a rotation of the device tip in the plane formed by t and p. We then define:

a′=t×p (6)

and

a=a′/la′l (7)

The vector a is normal to the desired plane of rotation for adjustment of the device. In the case where the remote navigation system is a magnetic navigation system, the external magnetic field is rotated about the vector a. In one embodiment, the amount of rotation could be a fixed step size as selected from a user interface. In another embodiment, the magnetic field could be rotated by an amount that depends on the length of the vector m, so that small lengths correspond to small adjustments and larger lengths correspond to larger adjustments, up to a predetermined threshold.

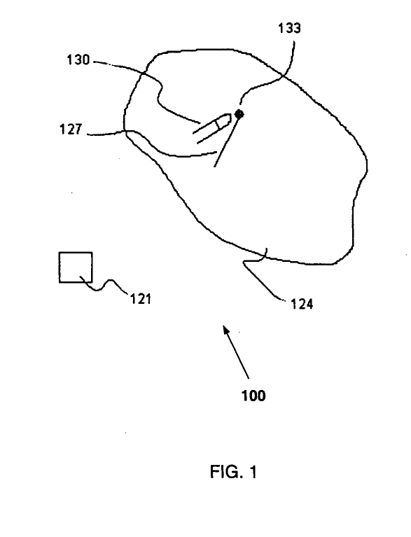

FIG. 1 is a view of a medical device near an anatomical surface within a subject's body, being controlled according to one embodiment of a method for adjusting the position of a medical device. An anatomical map in the form of a surface 124 is shown or displayed on a graphical display image. A mode button 121 is available to select the “adjustment” mode so that generation of the desired adjustment information can be enabled. A localized catheter 130 is also visible. A starting point 133 is selected by clicking or depressing a mouse button with a displayed cursor positioned in a location on the displayed image near the end of the medical device. In many cases, the starting point 133 can be at the catheter tip if the tip is very close to or is touching the tissue surface. The line 127 represents the movement of the mouse towards a desired adjustment direction. The line 127 is “rubber-banded” from the selected point to indicate the desired adjustment direction m₂ for adjustment of the medical device 130. Upon releasing the mouse button, appropriate device actuation controls are then computed as described above, and applied via the magnetic navigation system to provide intuitive adjustment of the device. This allows the user to control the device very easily and effectively, especially for fine adjustments of device positioning.

In an alternate embodiment, the “rubber-band” line 127 may be absent, and the appropriate movement direction of the mouse or other input device is taken as input to determine the appropriate actuation required.

In still another embodiment, the direction of movement implied by the user movement of the mouse or other input device could be directly used to control or actuate the device, without using any surface normal or other surface information, or even without user selection of a point on the surface. Thus, for example, in one embodiment in the case when a magnetic navigation system is used, the externally applied magnetic field could be simply rotated (with a suitable sense of rotation) about an axis defined as the perpendicular direction to the mouse movement in the plane of the display. In another embodiment in the case of a mechanically actuated navigation system, the mouse movement alone could be directly used as input to the control system in order to effect an appropriate change in device configuration so as to cause the device tip to generally move in the indicated direction.

The user can visually see the result of the adjustments made, since the display is updated as the real-time position of the medical device changes. The medical device is also visible on Fluoroscopic imaging systems, which are normally used with such procedures. Further confirmation of the medical device location is available to the physician from ECG signal data. Thus, the user can interactively and repeatedly adjust the device as needed until a desired position is reached. In some cases, such an intuitive method of “explorative” device control could be preferable to an automated, iteration-based method of driving a medical device to selected targets. For example, one application involves the creation of an RF ablation lesion line where it is desired to position the catheter and ablate at a sequence of closely-spaced points on the endocardial surface.

In one embodiment, the line 127 would disappear when the user releases the mouse button, for purposes of clarity. Alternatively, the most recently displayed line could remain on the display image as a visual guide. Alternatively, upon release of the mouse button, the display of the line could be selectively turned on or off by the user, from a menu button or other interface element.

It should be noted that the mouse in the above exemplary embodiments may be any appropriate user-input device, and the navigational system may include systems other than magnetic navigational systems that are capable of guiding a medical device through a subject's body. The medical device could be adjusted likewise by employing any one of various types of remote navigation systems, such as those based on mechanical, electrostrictive, hydraulic, magnetostrictive, or other device actuation technologies other than magnetic actuation systems.

The above concepts can be generally applied in different forms. For instance, the map could be derived from a three-dimensional pre-operative CT or Magnetic Resonance imaging scan. When the remote navigation system is interfaced with a localization system, the real-time location of the catheter can be graphically rendered together with the three-dimensional anatomical data on the remote navigation system. The device can be adjusted as described above, with all of the surface and other geometrical data being derived from the pre-operative data. In this case, the user interface for user control and adjustment of the medical device could be a display on the remote navigation system, and the user indicates the desired adjustment direction directly on a graphical window in the remote navigation system displaying the three-dimensional objects.

In another embodiment, the three-dimensional user-input device may comprise a stylus device, such as the Sensable™ Haptic Stylus. Such a stylus device could yield a current (computer) catheter tip location and orientation of a virtual catheter tip. In this case, the user can move the stylus in a sweeping arc, for example. This movement can be used directly to define the adjustment vector p and thence a change in a control variable such as a magnetic field could be effected using equations (6) and (7).

In yet another embodiment, in the absence of a direct connection to a localization system, a computational model of the device could yield a current (computed) catheter tip location and orientation of a virtual catheter tip. The above scheme for user-driven adjustments relative to three-dimensional image data such as a pre-operative surface or volume could be implemented in essentially the same manner, except that instead of a real-time localized catheter tip orientation t, a computed catheter tip orientation t_{c} is employed instead.

The foregoing description of methods for adjusting and fine control of medical device positioning provides non-limiting illustrative examples, and without departing from the spirit and scope of the above concepts, other similar methods and implementations can be derived from the teachings described herein by persons skilled in the art of remote navigation. The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method for controlling a remote surgical navigation system to adjustably move a medical device relative to an object being displayed in an image on a display device, the method comprising:
using a user-input device to select a point at or near the tip of a medical device depicted in an image on a two-dimensional display;
moving the user-input device in a desired adjustment direction on the displayed image;
determining a surface normal at a location corresponding to the point selected by the user;
determining an actuation of the remote navigation system based on the surface normal and user input of the desired adjustment direction.

2. The method of claim 1, where said determination of actuation of the remote navigation system corresponds to an estimation of reorientation of the tip of the medical device.

3. The method of claim 1 wherein the surface normal is generally perpendicular to the surface.

4. The method of claim 1 further comprising displaying a line on the displayed image which corresponds to the movement of the user-input device and the desired adjustment direction of the medical device.

5. The method of claim 1 where determination of actuation of the remote navigation system includes an estimation of a three dimensional vector corresponding to the user-defined adjustment direction on the two dimensional display.

6. The method of claim 2, where the remote navigation system is a magnetic navigation system.

7. The method of claim 2, where the remote navigation system is a mechanically actuated navigation system.

8. The method of claim 2, where the remote navigation system is actuated by electrostrictive means.

9. The method of claim 6, where the actuation includes a rotation of an externally applied magnetic field generated by the magnetic navigation system.

10. The method of claim 9 wherein the amount of magnetic field rotation is a fixed step size.

11. The method of claim 9 wherein the amount of magnetic field rotation depends on the magnitude of movement of the user input device.

12. The method of claim 1 wherein the image being displayed on the display is updated to show the real-time position of the medical device after an adjustment direction has been selected and submitted by a user.

13. The method of claim 1 wherein the tip of the catheter is very close to or touching the surface of the object.

14. A method for controlling a remote surgical navigation system to adjustably move a medical device relative to an anatomical surface being displayed in an image on a display device, the method comprising:
using a user-input device to select a point at or near the tip of a medical device depicted in an image on a two-dimensional display;
moving the user-input device a desired length in a desired adjustment direction relative to the point selected by the user on the displayed image;
determining an actuation of the remote navigation system based on the user input of the desired adjustment direction.

15. The method of claim 14, where said determination of actuation of the remote navigation system corresponds to an estimation of reorientation of the tip of the medical device.

16. The method of claim 15, where the remote navigation system is a magnetic navigation system.

17. The method of claim 15, where the remote navigation system is a mechanically actuated navigation system.

18. The method of claim 15, where the remote navigation system is actuated by electrostrictive means.

19. The method of claim 16, where the actuation includes a rotation of an externally applied magnetic field generated by the magnetic navigation system.

20. A method for controlling a remote surgical navigation system to adjustably move a medical device relative to an anatomical surface being displayed in an image on a display device, the method comprising:
using a user-input device to select a desired adjustment direction for the medical device relative to the displayed anatomical image;
determining an actuation of the remote navigation system based on the user input of the desired adjustment direction.

21. The method of claim 20, where said determination of actuation of the remote navigation system corresponds to an estimation of reorientation of the tip of the medical device.

22. The method of claim 21, where the remote navigation system is a magnetic navigation system.

23. The method of claim 21, where the remote navigation system is a mechanically actuated navigation system.

24. The method of claim 21, where the remote navigation system is actuated by electrostrictive means.

25. The method of claim 22, where the actuation includes a rotation of an externally applied magnetic field generated by the magnetic navigation system.

26. The method of claim 22, where the user input device is a keyboard.

27. The method of claim 22, wherein the amount of magnetic field rotation is a fixed step size.

28. The method of claim 22 wherein the amount of magnetic field rotation depends on the magnitude of movement of the user input device.

29. A method for controlling a remote surgical navigation system to adjustably move a medical device relative to an object being displayed in an image on a display device, the method comprising:
using a user-input device to select a point at or near the tip of a medical device depicted in an image on a two-dimensional display;
moving the user-input device in a desired adjustment direction relative to the point on the displayed image selected by the user;
determining a surface normal at a location corresponding to the point selected by the user;
determining a two-dimensional vector m corresponding to the direction and length of the movement of the user-input device;
determining a three-dimensional vector p in the tangent plane perpendicular to the surface normal, whose two-dimensional projection onto the two-dimensional displayed image yields the two-dimensional vector m; and
determining a rotation of the device tip in the plane formed by a vector t representing the device tip's initial orientation and the three-dimensional vector p, which rotation corresponds to the desired adjustment movement.

30. The method of claim 29 further comprising the step of displaying on the displayed image a line having a first endpoint corresponding to the user-selected point, and a direction and length corresponding to the length of movement of the user-input device in the desired adjustment direction.

31. The method of claim 30 further comprising applying a rotation matrix for transposing the two-dimensional vector m, representing the length and direction of the line displayed on the two-dimensional display, to a three-dimensional vector with respect to a known three-dimensional coordinate system relative to the object that is being display.
